(19) United States
(12) Patent Application Publication
(10) Pub. No.: US 2024/0240273 A1
(43) Pub. Date: Jul. 18, 2024

(54) Compositions and Methods for Detecting Hepatitis Delta Virus by a Dual-Target Assay

(71) Applicant: Roche Molecular Systems, Inc., Pleasanton, CA (US)

(72) Inventors: Kiran Adhikary, Pleasanton, CA (US); Rajiv Dua, Manteca, CA (US); Slav Dugenny, San Leandro, CA (US); Marintha Heil, Danville, CA (US); Calvin Mano, Berkeley, CA (US); Pauline Rivera Luis, Antioch, CA (US); Jingtao Sun, San Ramon, CA (US)

(21) Appl. No.: 18/558,830

(22) PCT Filed: May 4, 2022
(86) PCT No.: PCT/EP2022/061968

§ 371 Date: Nov. 3, 2023

Related U.S. Application Data
(60) Provisional application No. 63/185,176, filed on May 6, 2021.

(51) Int. Cl. C12Q 1/70; C12Q 1/6851

(52) U.S. Cl. CPC C12Q 1/706; C12Q 1/6851; C12Q 2600/16

(57) ABSTRACT

Methods for the rapid detection of the presence or absence of Hepatitis Delta Virus (HDV) in a biological or non-biological sample are described. The methods can include performing an amplifying step, a hybridizing step, and a detecting step. Furthermore, primers, probes targeting the HDV Ribozyme domain and the HDV Hepatitis Delta Antigen (HDAg) gene, along with kits are provided that are designed for the detection of HDV.

### FIELD OF THE INVENTION

The present disclosure relates to the field of molecular diagnostics, and more particularly to the detection of Hepatitis Delta Virus (HDV) by a dual-target polymerase chain reaction (PCR) assay.

### BACKGROUND OF THE INVENTION

Hepatitis Delta virus (HDV) is a satellite of hepatitis B virus (HBV) for transmission and propagation with an estimated global burden of 15-20 million. The HDV particle is 35-37 nm in diameter and has a ribonucleoprotein complex surrounded by an HBV envelope. The ribonucleoprotein is composed of a circular negative single-stranded viral RNA genome, approximately 1700 nucleotides, and two isoforms (Small and Large) of the HDV protein. Phylogenetic analysis of the full-length HDV genome classified HDV into eight genotypes and many sub-genotypes. HDV genotypes show specific geographic divergence and high sequence diversity of 20%-30% across genotypes and 15% within the sub-genotype.

Clinically, HBV/HDV dual infection carry higher risks for liver complications and increased mortality as compared to the HBV infection alone. To manage higher risk with HDV infection, it is recommended that patients with positive HDV serology undergo active HDV infection evaluation by HDV RNA detection. Scores of in-house and commercial reverse-transcription polymerase chain reaction assays have been developed to detect and quantify HDV RNA. However, there is a lack of standardization across assays. An international quality control study to assess various HDV RNA detection assays showed a less than stellar assay performance with only 13 labs out of 28 labs (46.3%) spread across 17 countries showing proper HDV quantification (see Le Gal et al., Hepatology, 2016, Vol. 64, No. 5, p. 1483-1494, and incorporated herein by reference in its entirety).

The main challenges associated with the robust HDV quantification include (a) high genetic diversity of HDV genome, (b) limited number of sequences with skewed temporal and spatial collection, and (c) HDV biology with high mutation rates combined with editing and recombination. Thus, there is a need in the art for a quick, reliable, specific, and sensitive method to detect all HDV genotypes and sub-genotypes.

### SUMMARY OF THE INVENTION

The present invention discloses a reverse-transcription polymerase chain reaction (RT-PCR) dual-target assay to detect and quantify HDV in blood, plasma or serum samples. The challenges associated with the robust HDV detection as discussed above was solved by detecting two HDV targets selected in-silico for high inclusivity, the Ribozyme domain and the Hepatitis Delta Antigen (HDAg) gene target regions on the HDV RNA genome. In comparison to current lab-developed and commercial HDV assays, which only detect single HDV targets, detection of two highly inclusive targets in this invention has advanced the field by providing an added benefit to properly detect and quantify HDV in case one target failed detection due to rapid virus evolution. Certain embodiments in the present disclosure relate to methods for the rapid detection of the presence or absence of HDV in a biological or non-biological sample, for example, multiplex detection of HDV by real-time RT-PCR in a single test tube. Embodiments include methods of detection of HDV comprising performing at least one cycling step, which may include an amplifying step and a hybridizing step. Furthermore, embodiments include primers, probes, and kits that are designed for the detection of HDV in a single tube. The detection methods are designed to target the Ribozyme domain and the Hepatitis Delta Antigen (HDAg) gene, which allows one to detect HDV in a single test.

In one aspect, a method for detecting at least two target nucleic acids of HDV in a sample is provided, including (a) providing a sample; (b) performing an amplification step comprising contacting the sample with at least two sets of primers to produce amplification products, if the at least two target nucleic acids of HDV are present in the sample; (c) performing a hybridization step, comprising contacting the amplification products, if the at least two target nucleic acids of HDV is present in the sample, with at least two probes; and (d) performing a detection step, comprising detecting the presence or absence of the amplification products, wherein the presence of one of the amplification products is indicative of the presence of HDV in the sample, and wherein the absence of the amplification product is indicative of the absence of HDV in the sample; and wherein the at least two sets of primers and the at least two probes comprise: (i) a first set of primers comprising a forward primer comprising or consisting of a nucleic acid sequence of SEQ ID NOs: 1-4 or any combination of SEQ ID NOs: 1-4; and a reverse primer comprising or consisting of a nucleic acid sequence of SEQ ID NOs: 5-6, or a combination thereof; and a first probe or a first set of probes comprising or consisting of a nucleic acid sequence of SEQ ID NOs: 7-8, or a complement thereof, or a combination of SEQ ID NOs: 7-8, or the complements thereof; and (ii) a second set of primers comprising a forward primer comprising or consisting of a nucleic acid sequence of SEQ ID NOs: 9-10, or a combination thereof; and a reverse primer comprising or consisting of a nucleic acid sequence of SEQ ID NOs: 11-12, or a combination thereof; and a second probe or a second set of probes comprising or consisting of a nucleic acid sequence of SEQ ID NOs: 13-15, or a complement thereof, or any combinations of SEQ ID NOs: 13-15, or the complements thereof. In a related embodiment, the sample is a biological sample. In another related embodiment, the biological sample is blood, plasma or serum.

In yet another embodiment, the first set of primers produces one or more amplification products of a first target nucleic acid that are detected by the first probe or the first set of probes and the second set of primers produces one or more amplification products of a second target nucleic acid that are detected by the second probe or the second set of probes. In one embodiment, the first target nucleic acid is the HDV Ribozyme domain and the second target nucleic acid is the HDV Hepatitis Delta Antigen (HDAg) gene.

In another aspect, a method for detecting HDV in a sample is provided, including (a) performing an amplifying step including contacting the sample with a one or more forward primers and one or more reverse primers specific for the HDV Ribozyme domain to produce amplification products of the Ribozyme domain, if HDV is present in the sample; and one or more forward primers and one or more reverse primers specific for the HDV Hepatitis Delta Antigen (HDAg) gene to produce amplification products of the HDAg gene if HDV is present in the sample; (b) performing a hybridizing step including contacting the Ribozyme domain amplification products with one or more detectable probes specific for the Ribozyme domain and contacting the HDAg gene amplification products with one or more detectable probes specific for the HDAg gene; and (c) detecting the presence or absence of the Ribozyme domain amplification products and/or the HDAg gene amplification products, wherein the presence of either the Ribozyme domain amplification products or the HDAg amplification products or both amplification products is indicative of the presence of HDV in the sample and wherein the absence of both the Ribozyme domain amplification products and the HDAg amplification products is indicative of the absence of HDV in the sample; wherein the one or more Ribozyme domain forward primers comprises or consists of a nucleotide sequence selected from SEQ ID NOs: 1-4, or any combinations of SEQ ID NOs: 1-4; and the one or more Ribozyme domain reverse primers comprises or consists of a nucleotide sequence selected from SEQ ID NOs: 5-6, or a combination thereof; and the one or more detectable Ribozyme domain probes comprises or consists of a nucleotide sequence selected from SEQ ID NOs: 7-8, or a complement thereof, or a combination of SEQ ID NOs: 7-8, or the complements thereof; and wherein the one or more HDAg gene forward primers comprises or consists of a nucleotide sequence selected from SEQ ID NOs: 9-10, or a combination thereof; and the one or more HDAg gene reverse primers comprises or consists of a nucleotide sequence selected from SEQ ID NOs: 11-12, or a combination thereof; and the one or more detectable HDAg gene probes comprises or consists of a nucleotide sequence selected from SEQ ID NOs: 13-15, or a complement thereof, or any combinations of SEQ ID NOs: 13-15, or the complements thereof.

In one embodiment, amplification of the Ribozyme domain target comprises using all forward primers comprising or consisting of a nucleotide sequence of SEQ ID NOs: 1-4, and both reverse primers comprising or consisting of a nucleotide sequence of SEQ ID NOs: 5-6, and detection of the Ribozyme domain amplification products comprises using both detectable probes comprising or consisting of a nucleotide sequence of SEQ ID NOs: 7-8, or the complements thereof. In another embodiment, amplification of the HDAg gene target comprises using both forward primers comprising or consisting of a nucleotide sequence of SEQ ID NOs: 9-10, and both reverse primers comprising or consisting of a nucleotide sequence of SEQ ID NOs: 11-12, and detection of the HDAg gene amplification products comprise using all detectable probes comprising or consisting of a nucleotide sequence of SEQ ID NOs: 13-15, or the complements thereof. In one embodiment, the sample is a biological sample. Specifically, the biological sample is blood, plasma or serum.

Other aspects provide an oligonucleotide comprising or consisting of a sequence of nucleotides selected from SEQ ID NOs: 1-20, or a complement thereof, which oligonucleotide has 50 or fewer nucleotides. In another embodiment, the present disclosure provides an oligonucleotide that includes a nucleic acid having at least 70% sequence identity (e.g., at least 75%, 80%, 85%, 90% or 95%, etc.) to one of SEQ ID NOs: 1-20, or a complement thereof, which oligonucleotide has 50 or fewer nucleotides. Generally, these oligonucleotides may be primer nucleic acids, probe nucleic acids, or the like in these embodiments. In certain of these embodiments, the oligonucleotides have 40 or fewer nucleotides (e.g., 35 or fewer nucleotides, 30 or fewer nucleotides, 25 or fewer nucleotides, 20 or fewer nucleotides, 15 or fewer nucleotides, etc.) In some embodiments, the oligonucleotides comprise at least one modified nucleotide, e.g., to alter nucleic acid hybridization stability relative to unmodified nucleotides. Optionally, the oligonucleotides comprise at least one label moiety and optionally at least one quencher moiety. In some embodiments, the at least one label moiety and the at least one quencher moiety are fluorescent moieties. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill in the art will recognize that individual substitutions, deletions or additions which alter, add or delete a single nucleotide or a small percentage of nucleotides (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

In one aspect, amplification can employ a polymerase enzyme having 5′ to 3′ nuclease activity. Thus, the label moiety and quencher moiety, which are the first and second fluorescent moieties, may be within no more than 8 nucleotides of each other along the length of the probe. In another aspect, the Ribozyme domain and/or the HDAg gene probes include a nucleic acid sequence that permits secondary structure formation. Such secondary structure formation generally results in spatial proximity between the first and second fluorescent moiety. According to this method, the second fluorescent moiety on the probe can be a quencher.

In one aspect, the Ribozyme domain and HDAg gene probes may be labeled with a fluorescent dye that acts as a reporter. The probe may also have a second dye that acts as a quencher. The reporter dye is measured at a defined wavelength, thus permitting detection and discrimination of the amplified HDV Ribozyme domain and HDAg gene targets. The fluorescent signal of the intact probes is suppressed by the quencher dye. During the PCR amplification step, hybridization of the probes to the specific single-stranded DNA template results in cleavage by the 5′ to 3′ nuclease activity of the DNA polymerase resulting in separation of the reporter and quencher dyes and the generation of a fluorescent signal. With each PCR cycle, increasing amounts of cleaved probes are generated and the cumulative signal of the reporter dye is concomitantly increased. Optionally, one or more additional probes (e.g., such as an internal reference control or other targeted probe (e.g., other viral nucleic acids) may also be labeled with a reporter fluorescent dye, unique and distinct from the fluorescent dye label associated with the Ribozyme domain and HDAg gene probes. In such case, because the specific reporter dyes are measured at defined wavelengths, simultaneous detection and discrimination of the amplified targets and the one or more additional probes is possible.

The present disclosure provides methods of detecting the presence or absence of HDV or HDV nucleic acid, in a biological sample from an individual. These methods can be employed to detect the presence or absence of HDV or HDV nucleic acid in biological samples such as serum, plasma, whole blood, liver tissue or other biological materials believed to have HDV present, for use in diagnostic testing. Additionally, the same test may be used by someone experienced in the art to assess other sample types to detect HDV or HDV nucleic acid. Such methods generally include performing a reverse transcription step and at least one cycling step, which includes an amplifying step and either a detectable probe binding step or a dye-binding step. Typically, the amplifying step includes contacting the sample with a plurality of pairs of oligonucleotide primers to produce one or more amplification products if a nucleic acid molecule is present in the sample, the probe binding step includes contacting the amplification product with one or more detectable probes specific for the amplification product, and the dye-binding step includes contacting the amplification product with a double-stranded DNA binding dye. Such methods also include detecting the presence or absence of binding of the double-stranded DNA binding dye into the amplification product, wherein the presence of binding is indicative of the presence of HDV or HDV nucleic acid in the sample, and wherein the absence of binding is indicative of the absence of HDV or HDV nucleic acid in the sample. A representative double-stranded DNA binding dye is ethidium bromide. Other nucleic acid-binding dyes include DAPI, Hoechst dyes, PicoGreen®, RiboGreen®, OliGreen®, and cyanine dyes such as YO-YO® and SYBR® Green. In addition, such methods also can include determining the melting temperature between the amplification product and the double-stranded DNA binding dye, wherein the melting temperature confirms the presence or absence of HDV or HDV nucleic acid.

In a further aspect, a kit for detecting one or more target nucleic acids of HDV is provided. In one embodiment, a kit for detecting a first target nucleic acid of HDV and a second target nucleic acid of HDV in a sample is provided, the kit comprising amplification reagents comprising: (a) a DNA polymerase having 5′ to 3′ nuclease activity; (b) nucleotide monomers; (c) a first set of primers and a first probe or a first set of probes for detecting the first target nucleic acid of HDV, wherein the first set of primers and the first probe or first set of probes comprise at least a forward primer comprising or consisting of a nucleic acid sequence of SEQ ID NOs: 1-4 and any combination of SEQ ID NOs: 1-4; and at least a reverse primer comprising or consisting of a nucleic acid sequence of SEQ ID NOs: 5-6, or a combination thereof; and the first probe or the first set of probes comprising or consisting of a nucleic acid sequence of SEQ ID NOs: 7-8, or a complement thereof, or a combination of SEQ ID NOs: 7-8, or the complements thereof; and (d) a second set of primers and a second probe or second set of probes for detecting the second target nucleic acid of HDV; wherein the second set of primers and the second probe or second set of probes comprise at least a forward primer comprising or consisting of a nucleic acid sequence of SEQ ID NOs: 9-10, or a combination thereof; and at least a reverse primer comprising or consisting of a nucleic acid sequence of SEQ ID NOs: 11-12, or a combination thereof; and the second probe or the second set of probes comprising a nucleic acid sequence of SEQ ID NOs: 13-15, or a complement thereof, or any combinations of SEQ ID NOs: 13-15, or the complements thereof. In one embodiment, the first target nucleic acid is the HDV Ribozyme domain and the second target nucleic acid is the HDV Hepatitis Delta Antigen (HDAg) gene. In a further embodiment, a kit for detecting one or more target nucleic acids of HDV is provided. The kit can include a plurality of sets of Ribozyme domain and/or HDAg gene primers specific for amplification of the Ribozyme domain target and/or the HDAg gene target; and one or more detectable Ribozyme domain and/or HDAg gene probes specific for detection of the respective amplification products. The kit can include probes already labeled with donor and corresponding acceptor fluorescent moieties, or can include fluorophoric moieties for labeling the probes. The kit can also include nucleoside triphosphates, nucleic acid polymerase, and buffers necessary for the function of the nucleic acid polymerase. The kit can also include a package insert and instructions for using the primers, probes, and fluorophoric moieties to detect the presence or absence of HDV in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows the relative locations of the primers and probes used for amplification and detection of the HDV Ribozyme domain target relative to GenBank Accession No. AF098261 (HDV Genotype 1).

FIG. 2 shows the relative locations of the primers and probes used for amplification and detection of the HDV HDAg gene target relative to GenBank Accession No. AF098261.

FIG. 3 shows the results of the experiment described in Example 4 to determine assay sensitivity using the WHO HDV NAT with 24 replicates each at 25, 10, 5, 2, 1, and 0.5 IU/mL level.

FIG. 4 shows the results of the experiment described in Example 4 to determine assay linearity in which HDV armored RNA (arRNA) was tested at 8 replicates per concentration between 100 copies/mL and 10¹⁰ copies/mL.

FIG. 5 shows the results of the experiment described in Example 4 to determine assay linearity in which HDV RNA from positive plasma samples was tested at 3 replicates per 10-fold dilution between 2.3E+00 IU/mL and 2.3E+05 IU/mL.

FIG. 6 shows the results of the experiment described in Example 4 to determine the inclusivity of Dual-Target Assay using in vitro transcribed HDV genotype 1-8 sequences for the Ribozyme domain and HDAg gene, as described in the Example 3. Each genotype was tested over a 4 log concentration range between 10³ copies and 10⁷ copies

### DETAILED DESCRIPTION OF THE INVENTION

Diagnosis of HDV infection by nucleic acid amplification provides a method for rapidly and accurately detecting the viral infection. A real-time reverse transcription polymerase chain reaction (RT-PCR) assay for detecting HDV in a sample is described herein. Primers and probes for detecting HDV are provided, as are articles of manufacture or kits containing such primers and probes. The increased sensitivity of real-time PCR for detection of HDV compared to other methods, as well as the improved features of real-time PCR including sample containment and real-time detection of the amplified product, make feasible the implementation of this technology for routine diagnosis of HDV infections in the clinical laboratory.

Hepatitis delta virus (HDV) is an infectious agent dependent upon hepatitis B virus (HBV) for the formation of viral particles. The HDV genome is a small single-stranded RNA of approximately 1700 nucleotides in length that is circular in conformation. The genome RNA is capable of folding using about 74% base pairing to form an unbranched rodlike structure. Replication of the HDV genome occurs through a symmetrical rolling-circle mechanism that involves RNA intermediates, and results in the accumulation of new genomes and complementary RNA species known as antigenomes. In a classic HDV infection, up to 300,000 copies of genome and 100,000 copies of antigenome accumulate per infected cell during HDV genome replication. It is believed that the genomic and antigenomic RNA circles act as templates for the generation of the multimeric strands of both polarities, which are greater than the 1700-nucleotide unit length. These are processed to unit length RNAs due to the presence of a site-specific ribozyme sequence in both the genome and antigenome. After ribozyme cleavage, the unit-length RNAs are ligated to form new circular RNA species. Since HDV does not encode its own replicase and can replicate autonomously in its host, one or more host RNA polymerases are redirected for its replication (Taylor and Pelchat, Future Microbiol 5:393-402, 2010).

A third HDV RNA species approximately 900 nucleotides in length and of antigenomic polarity is also produced at approximately 500 copies per infected cell in the classic HDVHBV infection. The open reading frame of this RNA encodes a protein that is 195 amino acids in length and is referred to as the small delta antigen (S-HDAg) and referred simply as HDAg in this disclosure. During replication, an adenosine deaminase that acts on dsRNA converts an adenosine in the termination codon of HDAg to an inosine. This amino acid conversion leads to the generation of an mRNA where the termination codon encodes tryptophan, resulting in the production of a second viral protein species that is 19 amino acids longer at the C-terminus, referred to as the large delta antigen (L-HDAg) (Taylor and Pelchat, Future Microbiol 5:393-402, 2010).

Extensive sequence analyses of numerous isolates have led to the classification of HDV in at least eight distinct clades with different geographic distributions. Genotype 1 is prevalent worldwide with other genotypes being endemic to different parts of the world. Genotype 2 is found in Southeast Asia, Taiwan, China and Japan. Genotype 3 is endemic to the Amazon Basin. Genotype 4 is found in Taiwan and Japan. Finally, genotypes 5 to 8 are prevalent in Africa.

The disclosed methods may include performing at least one cycling step that includes amplifying one or more portions of HDV Ribozyme domain nucleic acid target and HDV HDAg gene nucleic acid target from a sample using one or more pairs of Ribozyme domain primers and/or one or more pairs of HDAg gene primers. “Ribozyme domain primers” or “HDAg primers” as used herein refer to oligonucleotide primers that specifically anneal to nucleic acid sequence in the Ribozyme domain and the HDAg gene, respectively, and initiate DNA synthesis therefrom under appropriate conditions. Each of the discussed Ribozyme domain or HDAg gene primers anneals to a target within or adjacent to the respective target nucleic acid molecule such that at least a portion of each amplification product contains nucleic acid sequence corresponding to the target. The one or more of the Ribozyme domain amplification products and/or the HDAg gene amplification products are produced provided that one or more of the Ribozyme domain nucleic acid and/or the HDAg gene nucleic acid is present in the sample, thus the presence of these one or more of amplification products is indicative of the presence of HDV in the sample. The amplification product should contain the nucleic acid sequences that are complementary to one or more detectable probes for the Ribozyme domain or for the HDAg gene. Each cycling step includes an amplification step, a hybridization step, and a detection step, in which the sample is contacted with the one or more detectable probes for the Ribozyme domain or for the HDAg gene for detection of the presence or absence of HDV in the sample.

As used herein, the term “amplifying” refers to the process of synthesizing nucleic acid molecules that are complementary to one or both strands of a template nucleic acid molecule (e.g., HDV Ribozyme domain or HDV HDAg gene). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′—OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolo-pyrimidine analog, a pseudo-dU, a nitro pyrrole, a nitro indole, 2′-O-methyl Ribo-U, 2′-O-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference. Examples of these types of nucleic acid modifications are described in, e.g., U.S. Pat. No. 6,001,611, which is incorporated herein by reference.

The present disclosure provides methods to detect Hepatitis delta virus (HDV) by amplifying, for example, a portion of the HDV Ribozyme domain nucleic acid sequence and/or the HDV Hepatitis Delta Antigen (HDAg) gene nucleic acid sequence. Nucleic acid sequences of various genotypes and sub-genotypes of HDV are available (e.g., GenBank Accession Nos. AF098261 for Genotype 1, AF104264 for Genotype 2, AB037948 for Genotype 3, AB118820 for Genotype 4, AM183326 for Genotype 5, AM183332 for Genotype 6, AM183333 for Genotype 7, AM183330 for Genotype 8). Specifically, primers and probes to amplify and detect the Ribozyme domain and the HDAg gene nucleic acid molecule targets are provided by the embodiments in the present disclosure. For detection of HDV, primers and probes to amplify the Ribozyme domain and/or the HDAg gene are provided. HDV nucleic acids other than those exemplified herein can also be used to detect HDV in a sample. For example, functional variants can be evaluated for specificity and/or sensitivity by those of skill in the art using routine methods. Representative functional variants can include, e.g., one or more deletions, insertions, and/or substitutions in the HDV nucleic acids disclosed herein.

More specifically, embodiments of the oligonucleotides each include a nucleic acid with a sequence selected from SEQ ID NOs: 1-20, a substantially identical variant thereof in which the variant has at least, e.g., 80%, 90%, or 95% sequence identity to one of SEQ ID NOs: 1-20, or a complement of SEQ ID NOs: 1-20, and the variant.

TABLE I
Ribozyme Domain Primers and Probes

| SEQ | | | | |
|---|---|---|---|---|
| ID NO | NAME | DESCRIPTION | SEQUENCE | MODIFICATION |
| 1 | HDV1-GS2-FP1- | Forward primer | AACTGGCTCTCCCTTAGCCAT | t_BB_dC = |
| | TBB | | <t_BB_dC> | t-butylbenzyl-dC |
| 2 | HDV1-GS2-FP3- | Forward primer | GACTGGGCATTCCCTTTGCCAT | t_BB_dC = |
| | TBB | | <t_BB_dC> | t-butylbenzyl-dC |
| 3 | HDV1-GS3-FP2 | Forward primer | GGTGGATCTCCCTTTGCCATC | |
| 4 | HDV1-GS2-FP4- | Forward primer | AGCTGGCACTCCCTTAGCCAT | t_BB_dC = |
| | TBB | | <t_BB_dC> | t-butylbenzyl-dC |
| 5 | HDV1-R6M-TBB | Reverse primer | GTCGGCATGGCATCTCCA | t_BB_dC = |
| | | | <t_BB_dC> | t-butylbenzyl-dC |
| 6 | HDV1-R6M_2- | Reverse primer | GTCGGCATGGGATCTCCA | t_BB_dC = |
| | TBB | | <t_BB_dC> | t-butylbenzyl-dC |
| 7 | HDV1-P.1-N | Probe | <FAM_Thr>ATGC<BHQ_2>CCAGG | FAM_Thr = Dye |
| | | | TCGGACCGCGAGG<Phos> | BHQ-2-Quencher |
| | | | | Phos = phosphate |
| 8 | HDV1_P2_4_RD | Probe | <FAM_Thr>CTCC<BHQ_2>TTCG | FAM_Thr = Dye |
| | | | GATGCCCAGGTCGGACCACG | BHQ_2-Quencher |
| | | | <Phos> | Phos = phosphate |

TABLE 2
Hepatitis Delta Antigen (HDAg) Primers and Probes

| SEQ | | | | |
|---|---|---|---|---|
| ID NO | NAME | DESCRIPTION | SEQUENCE | MODIFICATION |
| 9 | CS-F1-M-TBB | Forward primer | CGGGCCGGCTGTTCTT<t_BB_dC> | t_BB_dC = |
| | | | | t-butylbenzyl-dC |
| 10 | CS-F2-M-TBB | Forward primer | CGGGCCGGCTACTCTT<t_BB_dC> | t_BB_dC = |
| | | | | t-butylbenzyl-dC |
| 11 | CS-R-TBB | Reverse primer | AAGGAAGGCCCTCGAGAAC | t_BB_dA = |
| | | | <t_BB_dA> | t-butylbenzyl-dA |
| 12 | CS-R2-NONTBB | Reverse primer | AAAGAAGGCCCTGGAGAACA | |
| 13 | CS-PROPN- | Probe | <FAM_Thr>CTTC<BHQ_2>TT<pdC | FAM_Thr-Dye |
| | PROBE1 | | ><pdC>T<pdC><pdC>CTG<pdC> | BHQ 2-Quencher |
| | | | TGA<Phos> | pdC = C5-propynyl-dC |
| | | | | Phos = phosphate |
| 14 | CS-PROPYN- | Probe | <FAM_Thr>CTTC<BHQ_2>TT<pdC | FAM_Thr = Dye |
| | PROBE2 | | ><pdC>T<pdC><pdC>TTG<pdC> | BHQ_2 = Quencher |
| | | | TGA<Phos> | pdC-C5-propynyl-dC |
| | | | | Phos = phosphate |
| 15 | CS-PROPYN- | Probe | <FAM_Thr>CTTC<BHQ_2>TT<pdC | FAM_Thr = Dye |
| | PROBE3 | | ><pdC>T<pdC><pdU>TGG<pdC> | BHQ 2-Quencher |
| | | | TGA<Phos> | pdC = C5-propynyl-dC |
| | | | | pdU = C5-propynyl-dU |
| | | | | Phos = phosphate |
| 16 | HDV-R3-D3- | Forward primer | GGGACGAAGCCGC<t_BB_dC> | t_BB_dC = |
| | FP1-TBB | | | t-butylbenzyl-dC |
| 17 | HDV-R3-D3- | Forward primer | GGCACGAAGCCCC<t_BB_dC> | t_BB_dC = |
| | FP2-TBB | | | t-butylbenzyl-dC |
| 18 | HDV-R3-D3- | Reverse primer | AAGAAGAGTAGCCGGCC | t_BB_dC = |
| | RP1-TBB | | <t_BB_dC> | t-butylbenzyl-dC |
| 19 | HDV-R3-D3- | Reverse primer | AAGAAGAACAGCCGGCC | t_BB_dC = |
| | RP2-TBB | | <t_BB_dC> | t-butylbenzyl-dC |
| 20 | HDV_R3_D3_PR1 | Probe | <FAM_Thr>CCGG<BHQ_2>GCG<p | FAM_Thr = Dye |
| | | | dC><pdU><pdC><pdC><pdC><pdC> | BHQ 2 = Quencher |
| | | | <pdU><pdC><Phos> | pdC = C5-propynyl-dC |
| | | | | pdU = C5-propynyl-dU |
| | | | | Phos-phosphate |

In one embodiment, the above-described sets of HDV Ribozyme domain and HDAg gene primers and probes are used in order to provide for detection of HDV in a biological sample suspected of containing HDV. The sets of primers and probes may comprise or consist the primers and probes specific for the Ribozyme domain or for the HDAg gene nucleic acid sequences, comprising or consisting of the nucleic acid sequences of SEQ ID NOs: 1-20. In another embodiment, the primers and probes for the Ribozyme domain and HDAg gene targets comprise or consist of a functionally active variant of any of the primers and probes of SEQ ID NOs: 1-15.

A functionally active variant of any of the primers and/or probes of SEQ ID NOs: 1-20 may be identified by using the primers and/or probes in the disclosed methods. A functionally active variant of a primer and/or probe of any of the SEQ ID NOs: 1-20 pertains to a primer and/or probe which provides a similar or higher specificity and sensitivity in the described method or kit as compared to the respective sequence of SEQ ID NOs: 1-20.

The variant may, e.g., vary from the sequence of SEQ ID NOs: 1-20 by one or more nucleotide additions, deletions or substitutions such as one or more nucleotide additions, deletions or substitutions at the 5′ end and/or the 3′ end of the respective sequence of SEQ ID NOs: 1-20. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule from the Ribozyme domain or from the HDAg gene nucleic acid sequences can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length). For example, oligonucleotide primers may be up to 30, 35, or 40 nucleotides in length. In addition to a set of primers, the methods may use one or more probes in order to detect the presence or absence of HDV. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to a HDV Ribozyme domain (target) nucleic acid and/or to a HDV HDAg gene (target) nucleic acid. A “probe” can be referred to as a “detection probe” meaning that it detects the target nucleic acid.

In some embodiments, the described Ribozyme domain and HDAg gene probes can be labeled with at least one fluorescent label. In one embodiment, the Ribozyme domain and HDAg gene probes can be labeled with a donor fluorescent moiety, e.g., a fluorescent dye, and a corresponding acceptor fluorescent moiety, e.g., a quencher. In one embodiment, the probe comprises or consists of a fluorescent moiety and the nucleic acid sequences comprise or consist of SEQ ID NO: 7, 8, 13, 14 and 15.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length. In some instances, oligonucleotide probes may be up to 30, 35, or 40 nucleotides in length.

Constructs containing HDV nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described HDV Ribozyme domain nucleic acid sequences (e.g., SEQ ID NOs: 1-6) and HDV HDAg gene nucleic acid sequences (e.g., SEQ ID NOs: 9-12). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described Ribozyme domain and HDAg gene nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ HDV nucleic acid such as RNA or DNA (cDNA). The template nucleic acid need not be purified; it may be a minor fraction of a complex mixture, such as HDV nucleic acid contained in human cells. HDV nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers (e.g., SEQ ID NOs: 1-6 and 9-12) are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target Ribozyme domain and/or HDAg gene nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times. FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the HDV target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range. As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced there between.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridine-isothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine×isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide, which contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

### Detection of Hepatitis Delta Virus

The present disclosure provides methods for detecting the presence or absence of Hepatitis Delta Virus (HDV) in a biological or non-biological sample. Methods provided avoid problems of sample contamination, false negatives, and false positives. The methods include performing at least one cycling step that includes amplifying a portion of the HDV Ribozyme domain and/or the HDV Hepatitis Delta Antigen (HDAg) gene target nucleic acid molecules from a sample using a plurality of pairs of Ribozyme domain and/or HDAg gene primers, and a FRET detecting step. Multiple cycling steps are performed, preferably in a thermocycler. Methods can be performed using the Ribozyme domain and/or HDAg gene primers and probes to detect the presence of HDV, and the detection of HDV Ribozyme domain and/or the HDV HDAg gene indicates the presence of HDV in the sample.

As described herein, amplification products can be detected using labeled hybridization probes that take advantage of FRET technology. One FRET format utilizes TaqMan® technology to detect the presence or absence of an amplification product, and hence, the presence or absence of HDV. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of HDV in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of HDV genomes). If amplification of HDV target nucleic acid occurs and an amplification product is produced, the step of hybridizing results in a detectable signal based upon FRET between the members of the pair of probes.

Generally, the presence of FRET indicates the presence of HDV in the sample, and the absence of FRET indicates the absence of HDV in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of an HDV infection.

Representative biological samples that can be used in practicing the methods include, but are not limited to blood, plasma, serum, liver samples, dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release HDV nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the Ribozyme domain and HDAg gene probes from the respective amplification products can confirm the presence or absence of HDV in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next. Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture or kits to detect HDV. An article of manufacture can include primers and probes used to detect HDV, together with suitable packaging materials. Representative primers and probes for detection of HDV are capable of hybridizing to HDV target nucleic acid molecules (e.g. the HDV Ribozyme domain and/or the HDV HDAg gene). In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to HDV target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the HDV Ribozyme domain and/or the HDV HDAg gene probes. Examples of suitable FRET donor fluorescent moieties and corresponding acceptor fluorescent moieties are provided above.

Articles of manufacture can also contain a package insert or package label having instructions thereon for using the HDV Ribozyme domain and/or the HDV HDAg gene primers and probes to detect HDV in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention.

### Example 1

### HDV Gene Targets

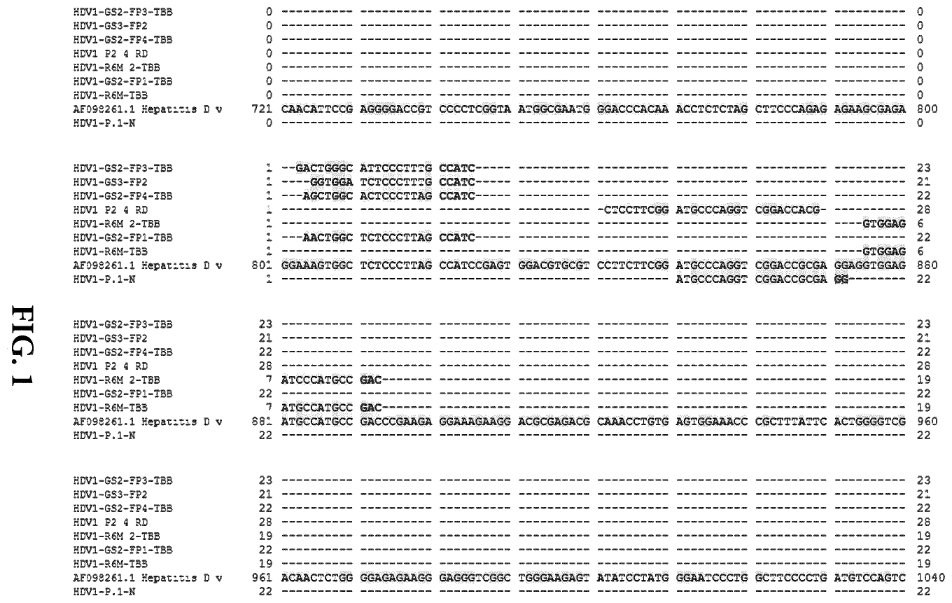

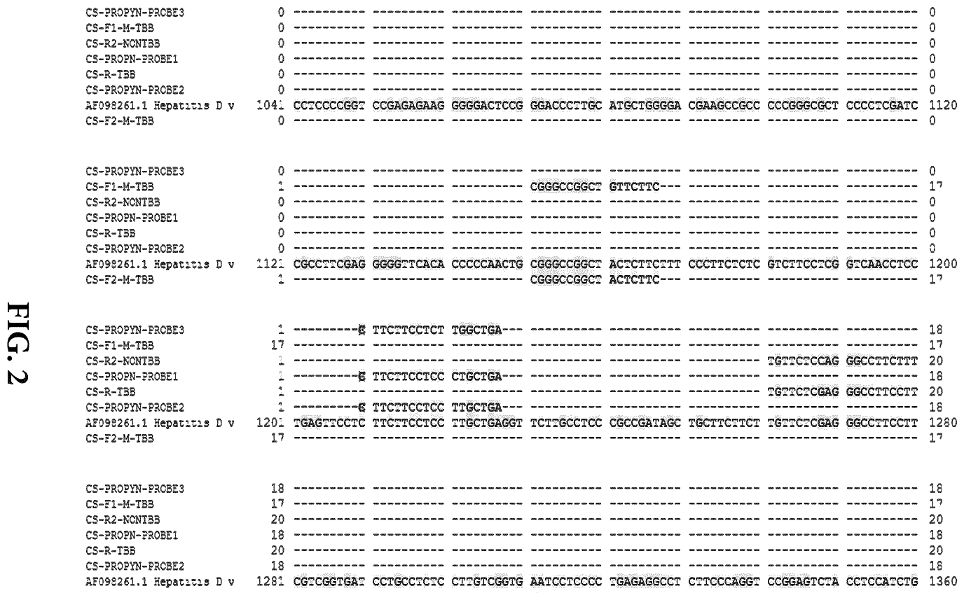

FIG. 1 shows the relative locations of the primers and probes used for amplification and detection of the HDV Ribozyme domain target relative to GenBank Accession No. AF098261 (HDV Genotype 1). FIG. 2 shows the relative locations of the primers and probes used for amplification and detection of the HDV HDAg gene target relative to GenBank Accession No. AF098261.

### Example 2

### PCR Experimental Conditions

Real-time PCR detection of the Ribozyme domain target or the HDAg gene were performed using the Cobas® 6800/8800 systems (Roche Molecular Systems, Inc., Pleasanton, CA). The final concentrations of the amplification reagents are shown below:

TABLE III
PCR Amplification Reagents

| Master Mix Component | Final Conc (50 uL) | |
|---|---|---|
| DMSO | 0-5.4 | % |
| NaN3 | 0.027-0.030 | % |
| Potassium acetate | 120.0 | mM |
| Glycerol | 3.0 | % |
| Tween 20 | 0.02 | % |
| EDTA | 0-43.9 | uM |
| Tricine | 60.0 | mM |
| Aptamer | 0.18-0.22 | uM |
| UNG Enzyme | 5.0-10.0 | U |
| Z05-SP-PZ Polymerase | 30.0-45.0 | U |
| dATP | 400.0-521.70 | uM |
| dCTP | 400.0-521.70 | uM |
| dGTP | 400.0-521.70 | uM |
| dUTP | 800.0-1043.40 | uM |
| Forward primer oligonucleotides | 0.20-0.40 | μM |
| Reverse primer oligonucleotides | 0.20-0.40 | μM |
| Probe oligonucleotides | 0.05-0.10 | μM |
| Manganese Acetate | 3.30-3.80 | mM |

The following table shows the typical thermoprofile used for PCR amplification reaction:

TABLE IV
PCR Thermoprofile

| | Target | Acquisition | Hold | Ramp Rate | | |
|---|---|---|---|---|---|---|
| Program Name | (° C.) | Mode | (hh:mm:ss) | (° C./s) | Cycles | Analysis Mode |
| Pre-PCR | 50 | None | 00:02:00 | 4.4 | 1 | None |
| | 94 | None | 00:00:05 | 4.4 | | |
| | 55 | None | 00:02:00 | 2.2 | | |
| | 60 | None | 00:06:00 | 4.4 | | |
| | 65 | None | 00:04:00 | 4.4 | | |
| 1st Measurement | 95 | None | 00:00:05 | 4.4 | 5 | Quantification |
| | 55 | Single | 00:00:30 | 2.2 | | |
| 2nd Measurement | 91 | None | 00:00:05 | 4.4 | 45 | Quantification |
| | 58 | Single | 00:00:25 | 2.2 | | |
| Cooling | 40 | None | 00:02:00 | 2.2 | 1 | None |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 3

### Materials and Methods

Samples: HDV armored RNA (arRNA), commercially obtained HDV positive plasma and serum samples, WHO HDV IS standard (PEI code number 7657/12), custom collected human volunteer donor HBV/HCV/HIV negative pooled plasma, and human volunteer donor HBV/HCV/HIV negative pooled serum (SeraCare) were used in the study. HDV positive serum and plasma samples were obtained from Boca Biologics.

HDV RNA assay: For assay design, Ribozyme and HDAg target regions on the HDV RNA genome were selected (FIGS. 1 and 2). HDV sequences were downloaded from Genbank, aligned, and genotype inclusive primers and probes designed. Sample volume of 0.5 mL was extracted on the Xobas® 6800 System (Roche Molecular Systems, Inc.). Extracted samples were eluted in 50 μL. 25 μL of sample was used for amplification using generic RNA mastermix and generic thermal profile on the Cobas® 6800 System. Data was analyzed using an algorithm testing framework (ATF) software with parameters custom optimized for HDV assay.

In-vitro RNA transcription: HDV sequences (genotype 1-8) were downloaded from Genbank. Sequences spanning the ribozyme and HDAg targets were cloned into the pSP6-polyA vector (Integrated DNA technologies, Inc.). Cloned sequences were verified by DNA sequencing. Plasmids with cloned sequences were used in the in-vitro transcription reaction using a SP6 promoter based MegaScript amplification kit (ThermoFisher). In-vitro RNA transcripts were purified by MegaClear kit (ThermoFisher) and copy number determined by HDV droplet digital PCR assay.

HDV assay performance studies: Assay linearity was determined using HDV arRNA and HDV positive plasma sample. WHO HDV NAT standard was used as a calibrator to report linearity in IU/mL. Linearity data was analyzed by polynomial regression. Assay sensitivity in terms of limit of detection (LOD) was determined using WHO HDV NAT standard with 24 replicates each at 25, 10, 5, 2, 1, and 0.5 IU/mL level. HBV/HCV/HIV negative pooled plasma was used as a control. LOD data was analyzed by probit. Assay specificity was determined using multiple replicates (n=92) of HBV/HCV/HIV negative pooled plasma and negative pooled serum.

In-silico Inclusivity and Exclusivity: HDV sequences detected in-silico by the HDV dual target assay were downloaded from Genbank, aligned, and phylogenetic analysis (bootstrap=100) was constructed using Geneious bioinformatics software (genious biologics). For exclusivity, HDV dual target assay primers and probes were aligned for cross-reactivity with HAV, HBV, HCV, HEV, and HIV sequences in the local database.

Plasma Separation card (PSC): Whole blood was collected in EDTA sample collection tubes. Samples such as HDV armored control, HDV plasma, and HDV WHO IS were spiked-in the whole blood at specified concentrations. To prepare PSC sample, 140 mL of whole blood (with or without spiked-in HDV) was spotted onto the 1 cm circles in the spotting area. After sample spotting, PSC were left at room temperature for 4 h and then kept in a ziplock bag with 4 g of desiccant. All bags with PSC were stored at various temperatures (ambient, −20° C., 45° C.) before analysis.

### Example 4

### Experimental Results

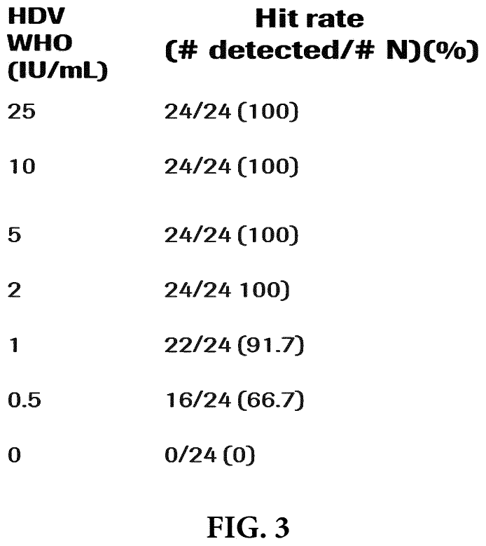

The performance of the Dual-Target Assay was assessed by several means. Determination of assay sensitivity was conducted using the WHO HDV NAT standard as described in Le Gal et al., Hepatology, 2016, Vol. 64, No. 5, p. 1483-1494, with 24 replicates each at 25, 10, 5, 2, 1, and 0.5 IU/mL level. FIG. 3 shows the results of this experiment with the Limit of Detection (LOD) determined from the Hit Rate and Probit Curve Fit to be 1.11 IU/mL.

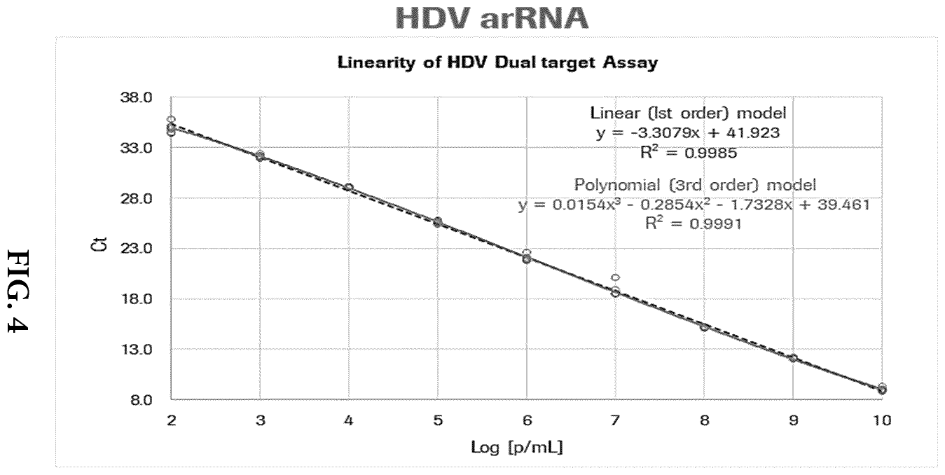

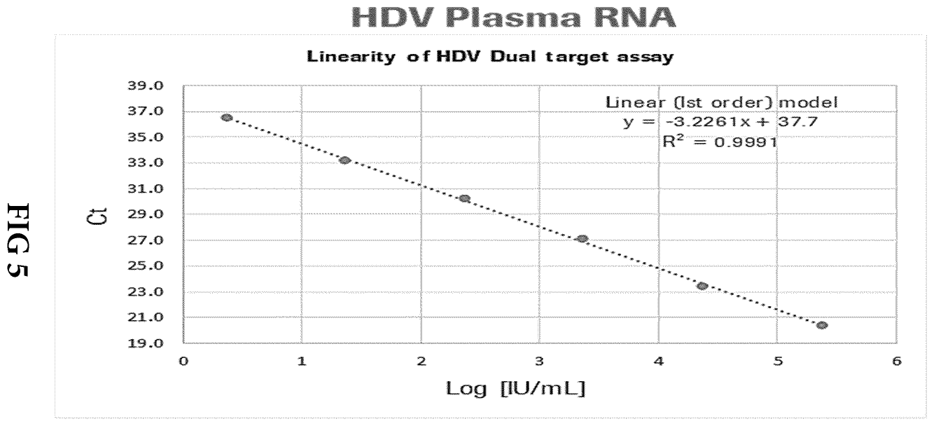

Assay linearity was determined using two types of samples. First, HDV armored RNA (arRNA) was tested at 8 replicates per concentration between 100 copies/mL and 10¹⁰ copies/mL. The results are shown on FIG. 4. Average Ct values ranged from 9.0 for 10¹⁰ copies/mL to 34.96 for 100 copies/mL and accurate quantification could be observed over a broad range of >8 logs. Next, HDV RNA from positive plasma samples was tested at 3 replicates per 10-fold dilution between 2.3E+00 IU/mL and 2.3E+05 IU/mL. As seen in FIG. 5, the average Ct values ranged between 20.38 and 36.49 and accurate quantification of HDV with clinical HDV samples could be observed over a broad range (>5 logs).

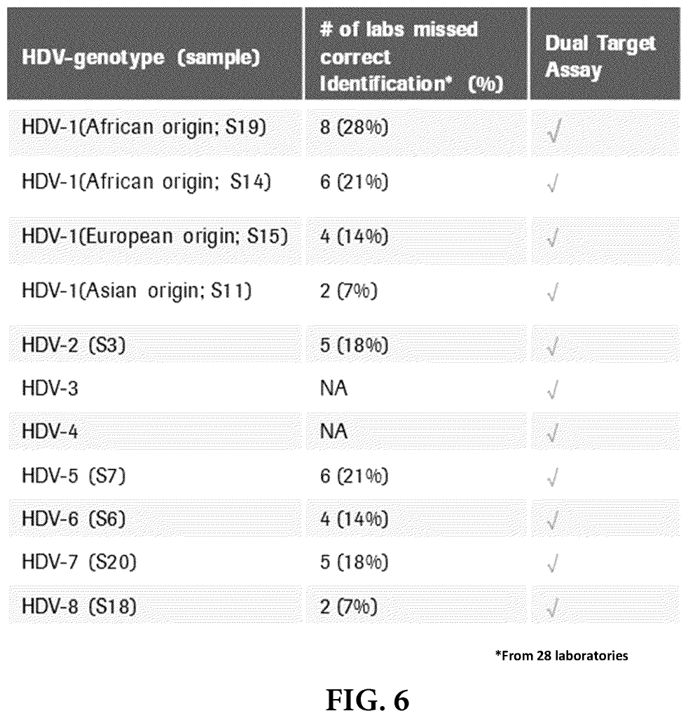

Experimental determination of the inclusivity of Dual-Target Assay was conducted using in vitro transcribed HDV genotype 1-8 sequences for the Ribozyme domain and HDAg gene, as described in the Example 3. Each genotype was tested over a 4 log concentration range between 10³ copies and 10⁷ copies. The results are shown on FIG. 6 and demonstrate that unlike many laboratory-developed HDV tests, the Dual-Target assay of the present invention was able to detect all eight HDV genotypes. In silico inclusivity and exclusivity studies were conducted and the results show that the Dual-Target Assay of the present invention would detect all HDV genotypes (1-8) but that HAV, HBV, HCV, HEV and HIV are predicted to not cross-react (data not shown).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for detecting at least two target nucleic acids of Hepatitis Delta Virus (HDV) in a sample, the method comprising:
(a) providing a sample;
(b) performing an amplification step comprising contacting the sample with at least two sets of primers to produce amplification products, if the at least two target nucleic acids of HDV are present in the sample;
(c) performing a hybridization step, comprising contacting the amplification products, if the at least two target nucleic acids of HDV are present in the sample, with at least two probes; and
(d) performing a detection step, comprising detecting the presence or absence of the amplification products, wherein the presence of one of the amplification products is indicative of the presence of HDV in the sample, and wherein the absence of the amplification products is indicative of the absence of HDV in the sample; and
wherein the at least two sets of primers and the at least two probes comprise:
(i) a first set of primers comprising at least one forward primer comprising a nucleic acid sequence of SEQ ID NOs: 1-4 or any combination of SEQ ID NOs: 1-4; and at least one reverse primer comprising a nucleic acid sequence of SEQ ID NOs: 5-6, or a combination thereof; and a first probe or first set of probes comprising a nucleic acid sequence of SEQ ID NOs: 7-8, or a complement thereof, or a combination of SEQ ID NOs: 7-8, or the complements thereof; and
(ii) a second set of primers comprising at least one forward primer comprising a nucleic acid sequence of SEQ ID NOs: 9-10, or a combination thereof; and at least one reverse primer comprising a nucleic acid sequence of SEQ ID NOs: 11-12, or a combination thereof; and a second probe or a second set of probes comprising a nucleic acid sequence of SEQ ID NOs: 13-15, or a complement thereof, or any combination of SEQ ID NOs: 13-15, or the complements thereof.

2. The method of claim 1, wherein:
the hybridizing step comprises contacting the amplification products with the two or more probes that are each labeled with a donor fluorescent moiety and a corresponding acceptor moiety; and
the detecting step comprises detecting the presence or absence of fluorescence resonance energy transfer (FRET) between the donor fluorescent moiety and the acceptor moiety of the probes, wherein the presence or absence of fluorescence is indicative of the presence or absence of HDV in the sample.

3. The method of claim 1, wherein the amplifying step employs a polymerase enzyme having 5′ to 3′ nuclease activity.

4. The method of claim 1, wherein the sample is a biological sample.

5. The method of claim 4, wherein the biological sample is blood, plasma or serum.

6. The method of claim 1, wherein the first set of primers produces one or more amplification products of a first target nucleic acid that are detected by the first probe or the first set of probes and/or the second set of primers produces one or more amplification products of a second target nucleic acid that are detected by the second probe or the second set of probes.

7. The method of claim 6, wherein the first target nucleic acid is the HDV Ribozyme domain and the second target nucleic acid is the HDV Hepatitis Delta Antigen (HDAg) gene.

8. A method for detecting Hepatitis D Virus (HDV) in a sample, the method comprising:
(a) performing an amplifying step comprising contacting the sample with a one or more forward primers and one or more reverse primers specific for the HDV Ribozyme domain to produce amplification products of the Ribozyme domain, if HDV is present in the sample; and one or more forward primers and one or more reverse primers specific for the HDV Hepatitis Delta Antigen (HDAg) gene to produce amplification products of the HDAg gene if HDV is present in the sample;
(b) performing a hybridizing step comprising contacting the Ribozyme domain amplification products with one or more detectable probes specific for the Ribozyme domain and contacting the HDAg gene amplification products with one or more detectable probes specific for the HDAg gene; and
(c) detecting the presence or absence of the Ribozyme domain amplification products and/or the HDAg gene amplification products, wherein the presence of either the Ribozyme domain amplification products or the HDAg amplification products or both amplification products is indicative of the presence of HDV in the sample and wherein the absence of both the Ribozyme domain amplification products and the HDAg amplification products is indicative of the absence of HDV in the sample;
wherein the one or more Ribozyme domain forward primers comprises a nucleotide sequence selected from SEQ ID NOs: 1-4, or any combination of SEQ ID NOs: 1-4; and the one or more Ribozyme domain reverse primers comprises a nucleotide sequence selected from SEQ ID NOs: 5-6, or a combination thereof; and the one or more detectable Ribozyme domain probes comprises a nucleotide sequence selected from SEQ ID NOs: 7-8, or a complement thereof, or a combination of SEQ ID NOs: 7-8, or the complements thereof; and
wherein the one or more HDAg gene forward primers comprises a nucleotide sequence selected from SEQ ID NOs: 9-10, or a combination thereof; and the one or more HDAg gene reverse primers comprises a nucleotide sequence selected from SEQ ID NOs: 11-12, or a combination thereof; and the one or more detectable HDAg gene probes comprises a nucleotide sequence selected from SEQ ID NOs: 13-15, or a complement thereof, or any combination of SEQ ID NOs: 13-15, or the complements thereof.

9. The method of claim 8, wherein the one or more Ribozyme domain forward primers comprise all four nucleotide sequences of SEQ ID NOs: 1-4, the one or more Ribozyme domain reverse primers comprise both nucleotide sequences of SEQ ID NOs: 5-6, and the one or more detectable Ribozyme domain probes comprise both nucleotide sequences of SEQ ID NOs: 7-8, or the complements, thereof.

10. The method of claim 8, wherein the one or more HDAg gene forward primers comprise both nucleotide sequences of SEQ ID NOs: 9-10, the one or more HDAg gene reverse primers comprise both nucleotide sequences of SEQ ID NOs: 11-12, and the one or more detectable HDAg gene probes comprise all three nucleotide sequences of SEQ ID NOs: 13-15, or the complements thereof.

11. The method of claim 8, wherein:
the hybridizing step comprises contacting the amplification products with the detectable probes that are each labeled with a donor fluorescent moiety and a corresponding acceptor moiety; and
the detecting step comprises detecting the presence or absence of fluorescence resonance energy transfer (FRET) between the donor fluorescent moiety and the acceptor moiety of the detectable probes, wherein the presence or absence of fluorescence is indicative of the presence or absence of HDV in the sample.

12. The method of claim 8, wherein the amplifying step employs a polymerase enzyme having 5′ to 3′ nuclease activity.

13. The method of claim 8, wherein the sample is a biological sample.

14. The method of claim 13, wherein the biological sample is blood, plasma or serum.

15. A kit for detecting a first target nucleic acid of HDV and a second target nucleic acid of HDV in a sample, the kit comprising amplification reagents comprising:
(a) a DNA polymerase having 5′ to 3′ nuclease activity;
(b) nucleotide monomers;
(c) a first set of primers and a first probe or first set of probes for detecting the first target nucleic acid of HDV,
wherein the first set of primers and the first probe or a first set of probes comprises at least a forward primer comprising a nucleic acid sequence of SEQ ID NOs: 1-4 and any combination of SEQ ID NOs: 1-4; and at least a reverse primer comprising a nucleic acid sequence of SEQ ID NOs: 5-6, or a combination thereof; and the first probe or the first set of probes comprising a nucleic acid sequence of SEQ ID NOs: 7-8, or a complement thereof, or a combination of SEQ ID NOs: 7-8, or the complements thereof; and
(d) a second set of primers and a second probe or a second set of probes for detecting the second target nucleic acid of HDV,
wherein the second set of primers and the second probe or second set of probes comprises at least a forward primer comprising a nucleic acid sequence of SEQ ID NOs: 9-10, or a combination thereof; and at least a reverse primer comprising a nucleic acid sequence of SEQ ID NOs: 11-12, or a combination thereof; and the second probe or the second set of probes comprising a nucleic acid sequence of SEQ ID NOs: 13-15, or a complement thereof, or any combinations of SEQ ID NOs: 13-15, or the complements thereof.

16. The kit of claim 15, wherein the first target nucleic acid is the HDV Ribozyme domain and the second target nucleic acid is the HDV Hepatitis Delta Antigen (HDAg) gene.

17. The kit of claim 15, wherein the first and second probes and the first set and second set of probes are labeled with a donor fluorescent moiety and a corresponding acceptor moiety.

18. An oligonucleotide comprising a sequence of nucleotides selected from SEQ ID NOs: 1-20, or a complement thereof, said oligonucleotide having 50 or fewer nucleotides.


SEQUENCE LISTING (20 sequences)

SEQ ID NO: 1 - DNA, 22 bp, Artificial Sequence
  HDV1-GS2-FP1-TBB Forward Primer
  modified_base (22)..(22): t-butylbenzyl-dC
aactggctctcccttagccatc

SEQ ID NO: 2 - DNA, 23 bp, Artificial Sequence
  HDV1-GS2-FP3-TBB Forward Primer
  modified_base (23)..(23): t-butylbenzyl-dC
gactgggcattccctttgccatc

SEQ ID NO: 3 - DNA, 21 bp, Artificial Sequence
  HDV1-GS3-FP2 Forward Primer
ggtggatctccctttgccatc

SEQ ID NO: 4 - DNA, 22 bp, Artificial Sequence
  HDV1-GS2-FP4-TBB Forward Primer
  modified_base (22)..(22): t-butylbenzyl-dC
agctggcactcccttagccatc

SEQ ID NO: 5 - DNA, 19 bp, Artificial Sequence
  HDV1-R6M-TBB Reverse Primer
  modified_base (19)..(19): t-butylbenzyl-dC
gtcggcatggcatctccac

SEQ ID NO: 6 - DNA, 19 bp, Artificial Sequence
  HDV1-R6M_2-TBB Reverse Primer
  modified_base (19)..(19): t-butylbenzyl-dC
gtcggcatgggatctccac

SEQ ID NO: 7 - DNA, 22 bp, Artificial Sequence
  HDV1-P.1-N Probe
  misc_feature: 5` FAM
  misc_feature: 3` phosphate
  misc_feature (4)..(5): BHQ-2 Quencher
atgcccaggtcggaccgcgagg

SEQ ID NO: 8 - DNA, 28 bp, Artificial Sequence
  HDV1_P2_4_RD Probe
  misc_feature: 5` FAM
  misc_feature: 3` phosphate
  misc_feature (4)..(5): BHQ-2 Quencher
ctccttcggatgcccaggtcggaccacg

SEQ ID NO: 9 - DNA, 17 bp, Artificial Sequence
  CS-F1-M-TBB Forward Primer
  modified_base (17)..(17): t-butylbenzyl-dC
cgggccggctgttcttc

SEQ ID NO: 10 - DNA, 17 bp, Artificial Sequence
  CS-F2-M-TBB Forward Primer
  modified_base (17)..(17): t-butylbenzyl-dC
cgggccggctactcttc

SEQ ID NO: 11 - DNA, 20 bp, Artificial Sequence
  CS-R-TBB Reverse Primer
  modified_base (20)..(20): t-butylbenzyl-dA
aaggaaggccctcgagaaca

SEQ ID NO: 12 - DNA, 20 bp, Artificial Sequence
  CS-R2-NONTBB Reverse Primer
aaagaaggccctggagaaca

SEQ ID NO: 13 - DNA, 18 bp, Artificial Sequence
  CS-PROPN-PROBE1 Probe
  misc_feature: 5` FAM
  misc_feature: 3` phosphate
  misc_feature (4)..(5): BHQ-2 Quencher
  modified_base (7)..(8): C5-propynyl-dC
  modified_base (10)..(11): C5-propynyl-dC
  modified_base (15)..(15): C5-propynyl-dC
cttcttcctccctgctga

SEQ ID NO: 14 - DNA, 18 bp, Artificial Sequence
  CS-PROPYN-PROBE2 Probe
  misc_feature: 5` FAM
  misc_feature: 3` phosphate
  misc_feature (4)..(5): GHQ-2 Quencher
  modified_base (7)..(8): C5-propynyl-dC
  modified_base (10)..(11): C5-propynyl-dC
  modified_base (15)..(15): C5-propynyl-dC
cttcttcctccttgctga

SEQ ID NO: 15 - DNA, 18 bp, Artificial Sequence
  CS-PROPYN-PROBE3 Probe
  misc_feature: 5` FAM
  misc_feature: 3` phosphate
  misc_feature (4)..(5): BHQ-2 Quencher
  modified_base (7)..(8): C5-propynyl-dC
  modified_base (10)..(10): C5-propynyl-dC
  modified_base (11)..(11): C5-propynyl-dU
  modified_base (15)..(15): C5-propynyl-dC
cttcttcctcttggctga

SEQ ID NO: 16 - DNA, 14 bp, Artificial Sequence
  HDV-R3-D3-FP1-TBB Forward Primer
  modified_base (14)..(14): t-butylbenzyl-dC
gggacgaagccgcc

SEQ ID NO: 17 - DNA, 14 bp, Artificial Sequence
  HDV-R3-D3-FP2-TBB Forward Primer
  modified_base (14)..(14): t-butylbenzyl-dC
ggcacgaagccccc

SEQ ID NO: 18 - DNA, 18 bp, Artificial Sequence
  HDV-R3-D3-RP1-TBB Reverse Primer
  modified_base (18)..(18): t-butylbenzyl-dC
aagaagagtagccggccc

SEQ ID NO: 19 - DNA, 18 bp, Artificial Sequence
  HDV-R3-D3-RP2-TBB Reverse Primer
  modified_base (18)..(18): t-butylbenzyl-dC
aagaagaacagccggccc

SEQ ID NO: 20 - DNA, 15 bp, Artificial Sequence
  HDV_R3_D3_PR1 Probe
  misc_feature: 5` FAM
  misc_feature: 3` phosphate
  misc_feature (4)..(5): BHQ-2 Quencher
  modified_base (8)..(8): C5-propynyl-dC
  modified_base (9)..(9): C5-propynyl-dU
  modified_base (10)..(13): C5-propynyl-dC
  modified_base (14)..(14): C5-propynyl-dU
  modified_base (15)..(15): C5-propynyl-dC
ccgggcgctcccctc